(19) United States
(12) Patent Application Publication
(10) Pub. No.: US 2023/0293804 A1
(43) Pub. Date: Sep. 21, 2023

(54) FLUID DISTRIBUTION DEVICE

(71) Applicant: Hospitech Respiration Ltd., Kfar-Saba (IL)

(72) Inventors: Noam WEIL, Hod-HaSharon (IL); Israel DEUTSCH, Petach-Tikva (IL); Yoav VENKERT, Zikhron-Yaakov (IL)

(73) Assignee: Hospitech Respiration Ltd., Kfar-Saba (IL)

(21) Appl. No.: 17/923,300

(22) PCT Filed: May 5, 2021
(86) PCT No.: PCT/IL2021/050517

§ 371 Date: Nov. 4, 2022

Related U.S. Application Data
(60) Provisional application No. 63/020,047, filed on May 5, 2020.

(51) Int. Cl. A61M 1/00; A61M 16/04

(52) U.S. Cl. CPC A61M 1/72; A61M 1/743; A61M 16/044; A61M 16/0463; A61M 2039/226; A61M 2205/128

(57) ABSTRACT

A fluid distribution device comprises a disposable enclosure, and disposable fluid connectors connectable to compatible panel connectors of a control system. The fluid distribution device can also comprise, within the enclosure, a two manifolds connected to each other via a plurality of fluid interconnectors each being controllable by a valve system. One manifold can comprise a fluid inlet for receiving fluid from one of the panel connectors, and the other manifold can comprise a plurality of fluid distribution ports adapted for establishing fluid communications between the fluid interconnectors and fluid lines external to the fluid distribution device.

### RELATED APPLICATION

This application claims the benefit of priority of U.S. Provisional Pat. Application No. 63/020,047 filed on May 5, 2020, the contents of which are incorporated herein by reference in their entirety.

### FIELD AND BACKGROUND OF THE INVENTION

The present invention, in some embodiments thereof, relates to fluid control, and, more particularly, but not exclusively, to a fluid distribution device. The fluid distribution device is particularly useful during mechanical ventilation by a tracheal tube.

Mechanical ventilation is necessary to assist patients having difficulties in breathing spontaneously. In the medical treatment of patients requiring breathing assistance, it is common to insert an endotracheal tube into the trachea of the patient, by way of the mouth, nose or any other surgically created opening, including tracheostomy. One end of the endotracheal tube is connected to a ventilator, which periodically forces air into the lungs through the tube. The distal end of the tube is typically provided with an inflatable cuff, which is inflated by conventional means subsequently to the insertion of the tube into the trachea. The inflated cuff is supposed to provides a seal against the interior wall of the trachea.

In order to ventilate a patient, air is forced into the patient’s lungs by a mechanical ventilation system. The forced air is transferred into the lungs from the ventilator via an endotracheal tube connected at its proximal side to the ventilator pipe, and its distal ends within the trachea above the carina. During exhalation, air flows back from the lungs trough the tube. The endotracheal tube is inserted into the trachea in order to maintain an open-air passage or to deliver oxygen and permit the suctioning of mucus from the lungs.

The length of the endotracheal tube is designed such that the proximal end of the tube is connected to a pipe attached to the ventilator, while the distal end of the tube is located within patient’s trachea, past the vocal cords above the carina.

Recently, the mechanical ventilation industry and the endotracheal tube industry have independently introduced new brands having advanced features that improve the treatment of intubated patients.

U.S. Published Application No. 20140366874, the contents of which are hereby incorporated by reference, discloses a system for controlling and monitoring flow in a cuffed endotracheal tube device. A connector panel has three or more connectors for establishing fluid communication with proximal ends of several lines of the endotracheal tube device. A processing unit instructs a control unit to execute various operations through the various lines.

### SUMMARY OF THE INVENTION

According to an aspect of some embodiments of the present invention there is provided a fluid distribution device. The fluid distribution device comprises: a disposable enclosure and disposable fluid connectors connectable to compatible panel connectors of a control system. The fluid distribution device can also comprise a first manifold and a second manifold, both mounted within the disposable enclosure and being connected to each other via a plurality of fluid interconnectors each being controllable by a valve system. The first manifold can comprise one or more fluid inlets for receiving fluid from one or more several panel connectors, and the second manifold can comprise a plurality of fluid distribution ports adapted for establishing fluid communications between the fluid interconnectors and external fluid lines.

According to an aspect of some embodiments of the present invention there is provided a fluid distribution device for mechanical ventilation. The fluid distribution device comprises: a disposable enclosure; disposable fluid connectors connectable to compatible panel connectors of a control system for controlling flow in a tracheal tube device. The fluid distribution device can also comprise a first manifold and a second manifold, both mounted within the disposable enclosure and being connected to each other via a plurality of fluid interconnectors each being controllable by a valve system. The first manifold can comprise a fluid inlet for receiving fluid from one of the panel connectors, and the second manifold can comprise a plurality of fluid distribution ports adapted for establishing fluid communications between the fluid interconnectors and fluid lines of the tracheal tube device.

According to some embodiments of the invention, the device second manifold is structured to established separate flow paths to at least two different distribution ports.

According to some embodiments of the invention, the separate flow paths comprise a first fluid path between a first pair of fluid interconnectors and a first distribution port, and a second fluid path between a second pair of fluid interconnectors and a second distribution port.

According to some embodiments of the invention, the second manifold is structured to established separate flow paths to at least three different distribution ports.

According to some embodiments of the invention the separate flow paths comprise a first fluid path between a first pair of fluid interconnectors and a first distribution port, a second fluid path between a second pair of fluid interconnectors and a second distribution port, and a third fluid path between an additional fluid interconnector and a third distribution port.

According to some embodiments of the invention, the disposable fluid connectors comprise a disposable cuff inflation connector connectable to a compatible control panel cuff inflation connector. According to some embodiments of the invention, the second manifold comprises a cuff inflation port for establishing a fluid communication between the disposable cuff inflation connector and a cuff inflation line of the tracheal tube device.

According to some embodiments of the invention, the disposable fluid connectors comprise a disposable vacuum connector connectable to a compatible control panel vacuum connector. According to some embodiments of the invention, the second manifold comprises a vacuum port for establishing a fluid communication between the disposable vacuum connector and a vacuum line generating under pressure in a waste collection container receiving waste fluid from a subject.

According to some embodiments of the invention, the first manifold comprises a waste port connectable to a waste line delivering the waste fluid to the waste collection container.

According to some embodiments of the invention, the first manifold comprises therein a first fluid channel and a second fluid channel separated from each other. According to some embodiments of the invention, the waste port of the first manifold is constituted to be fed by fluid from the second fluid channel, and is fluidly separated within the first manifold from the first fluid channel.

According to some embodiments of the invention, the first manifold comprises at least two separate fluid channels therein.

According to some embodiments of the invention, the disposable fluid connectors comprise a disposable fluid inlet connector connectable to a compatible gas supply panel connector of the control system. According to some embodiments of the invention, a first fluid channel is in fluid communication with the disposable fluid inlet connector, and wherein a second fluid channel is fluidly separated within the first manifold from the disposable fluid inlet connector.

According to some embodiments of the invention, the device comprises a saline inlet port connectable to saline supply line. According to some embodiments of the invention, the first fluid channel is constituted to be fed by saline from the saline inlet port, and wherein the second fluid channel is fluidly separated within the first manifold from the saline inlet port.

According to some embodiments of the invention, the device comprises a saline inlet port connectable to saline supply line. According to some embodiments of the invention, a first fluid channel is constituted to be fed by saline from the saline inlet port, and wherein a second fluid channel is fluidly separated within the first manifold from the saline inlet port.

According to some embodiments of the invention wherein the valve system is external to the fluid interconnectors.

According to some embodiments of the invention, the valve system is external to the disposable enclosure.

According to some embodiments of the invention, the valve system comprises a plurality of movable pressing members each aligned to engage a wall of one of the fluid interconnectors, such that a motion of a pressing member toward a respective wall generates a compressive force on the respective wall and restricts or ceases flow through a respective fluid interconnector.

According to some embodiments of the invention the valve system comprises a cam shaft having a plurality of cams each aligned with one of the pressing members in a manner that rotation of a cam establishes a linear motion of a respective pressing member.

According to an aspect of some embodiments of the present invention there is provided a method of distributing fluid within a tracheal tube device. The method comprises connecting the device as delineated above and optionally and preferably as further detailed below to a control system, and operating the controller of the control system to transmit control signals to the valve system.

According to an aspect of some embodiments of the present invention there is provided a system for controlling and monitoring flow in a cuffed tracheal tube device. The system comprises: a connector panel, adapted for connecting to a disposable enclosure of a fluid distribution device, and having a plurality of panel connectors adapted for connecting to disposable fluid connectors of the fluid distribution device; a valve system, configured for selectively controlling flow within the fluid distribution device; and a controller configured to transmit control signals to the valve system. According to some embodiments of the invention the fluid distribution device comprises a first manifold and a second manifold, both mounted within the disposable enclosure and being connected to each other via a plurality of fluid interconnectors each controllable by the valve system. According to some embodiments of the invention, the first manifold comprises a fluid inlet for receiving fluid from one of the panel connectors, and the second manifold comprises a plurality of fluid distribution ports adapted for establishing fluid communications between fluid interconnectors and fluid lines of the tracheal tube device.

According to an aspect of some embodiments of the present invention there is provided a method of distributing fluid within a tracheal tube device. The method comprises connecting a fluid distribution device to a control system controlling flow in a tracheal tube device; and operating a controller of the control system to transmit control signals to a valve system controlling flow within the fluid distribution device. The fluid distribution device optionally and preferably comprises: a disposable enclosure, and disposable fluid connectors connectable to compatible panel connectors of the control system. The fluid distribution device optionally and preferably comprises a first manifold and a second manifold, both mounted within the disposable enclosure and being connected to each other via a plurality of fluid interconnectors each being controllable by the valve system. According to some embodiments of the invention, the first manifold comprises a fluid inlet for receiving fluid from one of the panel connectors, and the second manifold comprises a plurality of fluid distribution ports adapted for establishing fluid communications between fluid interconnectors and fluid lines of the tracheal tube device.

According to some embodiments of the invention, the valve system is external to the disposable enclosure.

According to some embodiments of the invention, the disposable enclosure comprises a window exposing the fluid interconnectors to allow the valve system to engage the fluid interconnectors.

According to some embodiments of the invention, the first manifold comprises a waste port connectable to a waste line delivering a waste fluid to a waste collection container.

According to some embodiments of the invention, the disposable fluid connectors comprise a disposable vacuum connector connectable to a compatible control panel vacuum connector of the control system. According to some embodiments of the invention, the second manifold comprises a vacuum port for establishing a fluid communication between the disposable vacuum connector and a vacuum line generating under pressure in the waste collection container.

According to some embodiments of the invention, the control signals comprise a signal causing the valve system to open a fluid path from a fluid distribution port to the waste port.

According to some embodiments of the invention, the signal also causes the valve system to simultaneously open another fluid path from a panel connector of the control system to another fluid distribution port.

According to some embodiments of the invention, the control signals comprise a signal causing the valve system to open a fluid path from a panel connector of the control system to one of the interconnectors and from the interconnector to the waste port through another interconnector.

According to an aspect of some embodiments of the present invention there is provided a valve system. The valve system comprises: a plurality of flexible wall interconnectors, and a plurality of movable pressing members. Each movable pressing member is optionally and preferably aligned to engage a wall of one of the interconnectors, such that a motion of a pressing member toward a respective wall generates a compressive force on the respective wall and restricts or ceases flow through a respective interconnector. The valve system can also comprise a cam shaft having a plurality of cams each aligned with one of the pressing members in a manner that rotation of the cam establishes a linear motion of the pressing member.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings: FIG. 1 is a schematic illustration of a control system for controlling flow in a tracheal tube, and a fluid distribution device, according to some embodiments of the present invention;

FIGS. 2A and 2B are schematic illustrations of perspective external views of a back side (FIG. 2A) and a front side (FIG. 2B) of the fluid distribution device, according to some embodiments of the present invention;

FIG. 3 is a schematic illustration of the interior of the fluid distribution device, according to some embodiments of the present invention;

FIGS. 4A-D are schematic illustrations of a first manifold in accordance with some embodiments of the present invention and in greater detail, where FIG. 4A shows an isometric view, and FIGS. 4B-D show cross-sectional views along the lines A---A (FIG. 4B), A′---A′ (FIG. 4C) and A″---A″ (FIG. 4D) of FIG. 4A.

FIG. 5 is a is a schematic illustration of a representative example of separate flow paths within a manifold, according to some embodiments of the present invention;

FIGS. 6A-C are schematic illustrations of valve system, according to various exemplary embodiments of the present invention; and

FIGS. 7A-M are schematic illustrations showing several examples of fluid paths fluid distribution device, in accordance with some embodiments of the present invention.

### DESCRIPTION OF SPECIFIC EMBODIMENTS OF THE INVENTION

The present invention, in some embodiments thereof, relates to fluid control, and, more particularly, but not exclusively, to a fluid distribution device. The fluid distribution device is particularly useful during mechanical ventilation by a tracheal tube.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present embodiments comprise a fluid distribution device. The fluid distribution device is useful in many applications that require delivery of fluid to and from a cavity. Preferably, but not necessarily, the fluid distribution device is used in medical applications, in which case the cavity is an internal cavity of a mammal, e.g., a human subject. For example, the fluid distribution device can be used during mechanical ventilation, to provide fluid communications with fluid lines of a tracheal tube device. The fluid distribution device can alternatively be used to provide fluid communications with fluid lines connected to other parts of the body, such as the abdomen space, blood vessels or the bladder. The fluid distribution device can be used to deliver fluids to or from the cavity of hospitalized subjects, e.g., in the emergency room, during open surgery, etc.

When the fluid distribution device is employed during mechanical ventilation, and it can be used by a control system for controlling flow in a tracheal tube device, such as, but not limited to, an endotracheal tube device or a tracheostomy tube device.

When the fluid distribution device provides fluid communications with blood vessels or the bladder, it can aid in distributing fluids delivered to and from the body during various operations, such as, but not limited to, suctioning, pumping, rinsing, venting and the like.

As used herein “fluid” refers to any substance in a liquid or gaseous state.

A representative example of a control system with which the fluid distribution device can optionally be useful is the control system described in U.S. Published Application No. 20140366874 supra, the contents of which are hereby incorporated by reference. Such a control system can perform adjustable evacuation or suction of tracheal secretions, controlled rinsing and/or venting of the subglottal volume, and/or dynamical cuff sealing.

While the embodiments below are described with a particular emphasis to the use of the fluid distribution device, during mechanical ventilation, it is to be understood that the some embodiments of the invention use the fluid distribution device during other medical procedures and/or for other purposes.

It was found by the Inventor that it is beneficial to use the fluid distribution device in combination with a control system, since it reduces the maintenance complexity of the control system. In some embodiments of the present invention, the fluid distribution device is configured such that all the liquids that are extracted from the tracheal tube device pass through the fluid distribution device without passing through the control system. The advantage of these embodiments is that they eliminate the need to sterilize the control system between patients.

The fluid distribution device is optionally and preferably disposable in its entirety, so that following a use of the device with an endotracheal or tracheostomy tube device it can be discarded with other hospital wastes.

A control system for controlling flow in a tracheal tube device suitable for use with the fluid distribution device of the present embodiments is illustrated in FIG. 1. The control system is shown at 100 and the fluid distribution device is shown at 10.

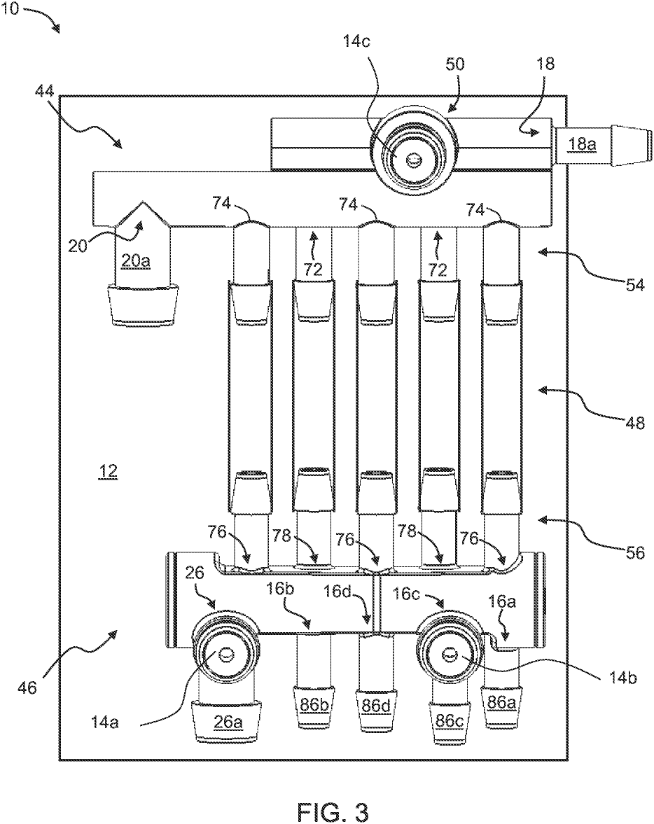

Device 10 comprises an enclosure 12 having disposable fluid connectors 14 and fluid distribution ports 16. Enclosure 12 is preferably removably mountable on system 100. A connector such as a barb (not shown, see, e.g., FIG. 3) is optionally and preferably mounted on each of ports 16. Device 10 can also comprise a saline inlet port 18, with a connector 18a mounted thereon, for connecting to a saline supply line 28 providing saline from a saline source 29. System 100 and device 10 are particularly useful for controlling and monitoring flow in a cuffed tracheal tube device 102 having a main lumen 202, a cuff 210, a cuff inflation line 106c, and one or more additional fluid lines 106a, 106b. Two or more of lines 106a-c, preferably each of lines 106a-c comprises a fluid connector (not shown) at its proximal end, which fluid connector being shape-wise and size-wise compatible with the connector of one of fluid distribution ports 16 to allow its connection to the respective the connector of the fluid distribution port fluid, hence to establish fluid communication between the respective line of device 102 and the respective fluid distribution port of device 10. The connector of one of the distribution ports (e.g., port 16d) can optionally and preferably be shape-wise and size-wise compatible with a fluid connector (not shown) at the proximal end of a retractable catheter 105 introducible into the main lumen 202 of the tracheal tube device 102.

The connectors mounted on one or more of the distribution ports 16 and the connectors at the proximal ends of lines 106a-c to be connected to distribution ports 16 are optionally and preferably provided with matching colors for preventing misconnections. As a representative example, which is not to be considered as limiting, the connector of port 16a can be provided with the same color as line 106a so that in use of device 10 the proximal end connector of line 106a is connected to the connector of port 16a, the connector of port 16b can be provided with the same color as line 106b so that in use of device 10 the proximal end connector of line 106b is connected to the connector of port 16b, the connector of port 16c can be provided with the same color as line 106c so that in use of device 10 the proximal end connector of line 106c is connected to the connector of port 16c, and the connector of port 16d can be provided with the same color as line 105 so that in use of device 10 the proximal end connector of line 105 is connected to the connector of port 16d.

System 100 and device 10 are suitable for use in conjugation with device 102 during any intubation procedure, including, without limitation, oral, nasal endotracheal intubation and tracheotomy.

In some embodiments of the present invention device 10 comprises a waste port 20 with a connector 20a connectable to a waste line 22 delivering waste fluid to a waste collection container 138, such as a trap bottle, having an under-pressure therein. In these embodiments device 10 optionally and preferably comprises a vacuum port 26, with a connector 26a which can be connected to clean line 24 leading from an outlet 139 collection container 138. Outlet 139 may be provided with a mechanism, such as floating member (not shown) for blocking waste fluid from flooding outlet 139, as known in the art.

A more detailed description of the principles and operation of device 10 is provided hereinafter.

System 100 comprises a connector panel 108 having several panel connectors 132. Each of panel connectors 132 is optionally and preferably shape-wise and size-wise compatible with one of the disposable fluid connectors 14 of device 10, such that when device 10 is mounted on connector panel 108 the connectors of device 10 engage and fittedly connect to the respective connectors of panel 108. Connector panel 108 can also include further additional connectors, for other operations, typically, but not exclusively, those performed using an under-pressure, such as, but not limited to, teeth brushing used for antiseptic and for evacuation of secretions from the oropharynx. Panel 108 can optionally and preferably comprise additional connectors for connecting various filters, including, without limitation, an antibacterial filter and a humidity filter. One or more of the connectors on panel 108 are optionally and preferably controlled by an electro-optical switch (not shown), which can be configured to alert in case of disconnection.

Connectors 132 can include a gas (e.g., air) supply connector 172 for providing airflow, for example, for the purpose of venting the subglottal volume or the drain collection container 138. In FIG. 1, gas supply connector 172 is shape-wise and size-wise compatible with disposable fluid connector 14c.

Connectors 132 can additionally include a vacuum connector 176 for connecting device 10 to an external under-pressure line or a vacuum pump, for example, via the vacuum network of a hospital or the like. Alternatively, system 100 can include a pump for providing vacuum conditions. The vacuum level is typically expressed in units of pressure, wherein lower pressure corresponds to higher vacuum level, and higher pressure corresponds to lower vacuum level. In FIG. 1, vacuum connector 176 is shape-wise and size-wise compatible with disposable fluid connector 14a.

Optionally, connectors 132 also include a cuff inflation connector 174 for providing cuff inflation fluid (e.g., air, saline), to be exclusively used for inflating cuff 210 through cuff inflation line 106c. In these embodiments, one of fluid distribution ports 16 of device 10, e.g., port 16c, is optionally and preferably in direct fluid communication with the disposable fluid connector of device 10 that connects to cuff inflation connector 174 (disposable connector 14b in FIG. 1), and is connected, in use of device 10, to the proximal end connector of line 106c.

System 100 optionally and preferably comprises a power supply 112, which can be a medical grade isolated power supply. Alternatively, system 100 can be connected to an external power supply unit (not shown). The power is preferably distributed to the components of the system via a power distributor board 114, which provide each component with the appropriate voltage. In some embodiments of the present invention system 100 comprises an Uninterruptible Power Supply (UPS) for ensuring continuous operation of system 100, e.g., when there is a need to disconnect the system from the main power supply for short periods or there is a shutdown of the main power. Any type of UPS can be used, for example, one or more series of Li-Ion cells, or the like.

System 100 preferably comprises a processing system 116 and a control system 118. Control system can comprise a main controller 120 and one or more operational modules 122. Controller 120 and operational modules 122 optionally and preferably have electronic circuits, wherein the electronic circuit of controller 120 is configured to control operational the electronic circuits modules 122 responsively to signals received from processing system 116. System 116, 118 and 120 can be provided as separate system units, as illustrated in FIG. 1, or two or more of these systems can be united to a single unit. Thus, for example, main controller 120 of system 118 can have also processing capabilities, in which case system 118 also functions as system 116, and system 100 does not include a separate processing system. Below, a reference to processing unit 116 encompasses both embodiments in which unit 116 is a separate system and embodiments in which system 118 serves also as a processing system.

Processing system 116 can be a general-purpose processor or a dedicated circuitry, and is configured to instruct the control system to execute various operations described herein. The operations are based on an algorithm, which can be supplemented to processing system 116. For example, the algorithm can be written onto a tangible computer readable medium, such as optical, magnetic or non-volatile electronic memory accessible by processing system 116. Processing system 116 can also receive signals from system 118. In these embodiments, processing system 116 analyzes the signals conveyed by system 118 in order to extract information from the signal. Based on the extracted information, system 118 carries on the operations.

The extracted information can also be displayed on a display device 124 or transmitted via an I/O communication panel 126. Panel 126 can include one or more communication ports, such as Universal Serial Bus (USB) ports to allow connection of system 100 to an external device such as an external computer, an external hard drive, an external memory medium, an external monitor, an external personal device (e.g., personal digital assistance (PDA), smart phone, etc.) or any other device capable of communicating via a communication port such as a USB port. Panel 126 can include an RS232 connection for connecting to an external monitor. Other types of connectors, such as PS2 ports and LAN ports, are also contemplated. Panel 126 serves for allowing the respective external device to download or upload data from and to system 100. Representative examples for data, which can be uploaded to system 100, include, without limitation, software updates for processing system 116 and history data of a particular subject. Representative examples for data which can be downloaded from system 100 include, without limitation, parameters detected and/or calculated by processing system 116.

Thus, processing system 116 is optionally and preferably configured for communicating with other processing units or computers. Such communication can be wired communication, e.g., via USB ports or the like, and/or it can be wireless communication, e.g., a Bluetooth® or a WiFi communication that can establish connection between system 116 and a remote location, for example, via the Internet. When the communication is wireless, panel 126 comprises a suitable wireless communication device for establishing such communication.

System 116 can also be configured for communicating (via wired or wireless communication) with systems which are capable of receiving and processing data but may also have other functions. Representative examples include, without limitation, a cellular telephone with data processing functionality, a personal digital assistant (PDA) with data processing functionality, a portable email device with data processing functionality (e.g., a BlackBerry® device), a portable media player with data processing functionality (e.g., an Apple iPod®), a portable gaming device with data processing functionality (e.g., a Gameboy®), and a tablet or touch screen display device with data processing functionality (e.g., an Apple iPad®). System 116 can be configured to receive data from and transmit data to any of these systems.

Processing system 116 instructs control system 118 to execute operations, via main controller 120, according to program instructions corresponding to a dedicated algorithm devised by the inventors of the present invention. Processing system 116 is preferably also configured for recording on a memory medium data calculated by system 116 or received from controller 120 or from an external source (via panel 126). Representative of parameters that can be recorded by system 116 include, without limitation, events chronology, cuff pressure, cuff leak, occlusion of one or more of the fluid lines, lung compliance, lung resistance, alerts, and one or more statistical analysis reports in the frequency or time domains pertaining to monitored cuff pressure control, coughing and other events.

The recorded data can be displayed on display device 124, for example, upon a specific request by the operator. The recorded data can also be transmitted or downloaded to an external system having processing capabilities. The recorded data can also be used by system 116 or the physician observing the data in various calculations or estimations that may require use of prerecorded data. For example, the recorded data can be used for analyzing trends of lungs compliance and resistance, thereby reducing the risk of abnormal lung conditions (e.g., acute respiratory distress syndrome, acute lung injury, ventilator-associated lung injury). Such analysis can include calculation of the resistance to flow in the main lumen of device 102 based on the history of tracheal and ventilator pressures. The resistance to flow is typically expressed in pressure units which describe the pressure drop along the length of the main lumen device 102. The resistance to flow history can be used, to calculate lungs compliance in order to alert development of Ventilator Associated Lung Injuries.

System 116 can monitor and operate controller 120, display data via display 124, handle a Graphic User Interface (GUI) displayed on display 124, log the operations and alerts of system 100, and/or transmit and receive data from external devices via I/O communication panel 126. The data can be displayed on display 124 graphically and/or in an alphanumeric presentation. System 116 is preferably configured to display visual messages, such as warning messages and system status messages via display 124. In some embodiments of the present invention, system 100 includes an electroacoustic device 130, such as a loudspeaker or buzzer, for generating acoustic signals responsively to electrical signals from processing system 116. For example, system 116 can be configured to accompany a warning message by an alarm signal.

Controller 120 and/or system 116 can optionally and preferably receive input also from a user interface system 128. System 128 can include operational buttons and/or touch screen for allowing the operator to insert subject details, select operations profiles, select display modes, change profiles parameters, and react to alerts and the like. System 128 is preferably configured to allow the operator to force procedures and/or to bypass procedures dictated by system 116. In various exemplary embodiments of the invention display 124 can be provided as a touch screen so as to allow display 124 also to serve as a user interface system, either in addition to or as an alternative to operational buttons that system 128 may or may not include.

System 100 preferably also comprises a valve system 180 controlled by the controller 120. Valve system 180 allows selective fluid communication between the distribution ports 16 and the connectors on panel 108, and optionally and preferably also between distribution ports 16 and the saline inlet port 18, and the waste port 20, in accordance with the procedures performed by controller 120. Preferably, valve system 180 is devoid of any contact with the fluids passing through the respective ports. In some embodiments of the present invention valve system 180 is external to device 10. For example, enclosure 12 of device 10 can be provided with a window 30 through which valve system 180 engages and controls fluid interconnectors in device 10, as described in greater detail below.

In some embodiments of the present invention the control system 118 execute, based on the algorithm and using, inter alia, valve system 180, operations including at least one of a rinsing procedure, a suctioning procedure, a cuff inflation procedure, a leak detection procedure and a venting procedure. This can be achieved by providing system 118 with at least one of: a rinsing module 152 for executing the rinsing operation, a suctioning module 154 for executing the suctioning operation, a cuff inflation module 156 for executing the cuff inflation operation, a leak detection module 158 for executing the leak detection operation and a venting module 160 for executing the venting operation.

In some embodiments of the present invention system 100 comprises an additional suctioning module 155 which generally serves for automatically removing lung secretions from the lower part of the trachea near or at the lungs. It is to be understood that although modules 154 and 155 are shown as separate modules, this need not necessarily be the case, since, for some applications, it may not be necessary for the suction operations above and/or below the cuff to be perfumed by separate modules. Thus, in some embodiments, module 154 performs also the operations described herein with respect to module 155. In these embodiments, system 100 optionally includes only one suctioning module 154.

For any of the rinsing operation, the suctioning operation, the leak detection operation and the venting operation, system 118 optionally and preferably automatically signal device 10 to select a fluid line from lines 106a and 106b and performs the respective operation through the selected line.

Modules 152, 154, 155, 156 and 160 can perform operations as described in U.S. Published Application No. 20140366874 supra, the contents of which are hereby incorporated by reference. Some operation principles of the modules of system 118 will now be explained.

Suctioning module 154 can comprise an adjustable pressure regulator and/or a flow meter (not shown). The pressure regulator controls the suctioning vacuum applied by module 154, and the flow meter measures the flow generated by the suctioning operation. Regulating the suctioning vacuum is advantageous since it allows suctioning secretions at improved efficiency with reduced risk of tissue damage. Measuring the flow is advantageous since it allows determining whether or not the respective fluid line is occluded and optionally the level (e.g., percentage) of occlusion. This is preferably performed by processing system 116 which receives, via controller 120, flow rate data from module 154 and determines, based on the flow rate data, whether or not there is an occlusion and optionally also the level of occlusion. Such determination can include thresholding.

Optionally, suctioning module 154 performs suction operation also below the cuff for evacuating secretions as further detailed hereinbelow.

Optionally, suctioning module 154 is also operated manually, for example, for oropharynx evacuation of secretions using a separate suction catheter introduced externally to main lumen of device 102 and/or for supplying an under-pressure for a teeth brushing device for teeth brushing while evacuating secretions from the oropharynx.

Rinsing module 152 optionally can comprise a pump and is configured to introduce a rinsing fluid into one of lines 106a and 106b. Rinsing module 152 is controlled by controller 120 which establishes a fluid communication between rinsing module 152 and one of lines 106a and 106b (for example, by signaling a valve system 180) and thereafter signals rinsing module 152 to pump the rinsing fluid. Controller 120 can select any of lines 106a and 106b for the rinsing operation. During the ventilation of a subject, there is preferably at least one time period at which controller 120 selects line 106a and at least one time period at which controller 120 selects line 106b for the rinsing operation.

The rinsing fluid can be of any type, including, without limitation, a liquid, which comprises an antiseptic substance, a biomarker substance, a local analgesic substance, and/or a secretion diluting substance.

Cuff inflation module 156 can comprise one or more pressure sensors (not shown) which monitor the pressure within cuff inflation line 106c (hence also within the cuff). Optionally and preferably there is more than one pressure sensor for safety and for increasing the accuracy of measurement. For example, module 156 can include two sensors and processing system 116 can compare the pressure values measured by the two sensors and issue an alert when the values are not consistent (e.g., deviate from each other by 10% or more). Module 156 also comprises a miniature pump, which forces filling fluid (typically air) into the cuff inflation line 106c, and one or more valves which control the amount and rate of fluid delivery into line 106c or deflate the cuff at a controlled rate.

Leak detection module 158 aids in assessing the level of sealing provided by the cuff. This can be done in more than one way, e.g., as described in U.S. Pat. No. 6,843,250 and U.S. Published Application No. 20090229605, both assigned to the same assignee as the present application and being incorporated by reference in their entirety as if fully set forth herein. Irrespectively of the technique employed by module to detect leakage, main controller 120 preferably receives leak detection data from module 158 and operates cuff inflation module 156 responsively to the data. For example, controller 120 can instruct module 156 to increase the pressure in the cuff when the data from module 158 indicates that a leaking duct has been formed, and to reduce the pressure in the cuff when the data indicates that the cuff provides adequate sealing.

In some embodiments of the present invention controller 120 monitors variations in the cuff pressure and operate cuff inflation module 156 responsively to the monitored pressure. It was found by the present inventor that the subject’s breathing may affect the intra cuff pressure. Therefore, the pressure variation pattern can be analyzed in terms of the periodicity and/or amplitude in order to maintain adequate sealing without over-inflating the cuff. According to some embodiments of the present invention when the amplitude of the cuff pressure variation is above a predetermined pressure threshold (typically from about 14 mmHg to about 16 mmHg, e.g., about 15 mmHg) for a time period which is above a predetermined time threshold (typically from about 15 minutes to about 25 minutes, e.g., about 20 minutes), controller 120 instructs models 156 to reduce the cuff pressure, preferably gradually.

The correlation between the variations in cuff pressure and breathing cycle of the subject is optionally and preferably utilized for detecting occlusion also in the cuff inflation line. In these embodiments, controller 120 provides processing system 116 with monitoring data pertaining to the cuff pressure as a function of time. System 116 analyzes the data and extracts respiratory features from the data as further detailed hereinunder. In was found by the present inventor that lack of correlation between the cuff pressure variation and the respiratory rate, indicates that the cuff inflation line is occluded. Thus, according to some embodiments of the present invention when the system 116 is unable to extract respiratory feature from the cuff pressure data, or when the extracted features do not correlate with a respiratory rate within a predetermined threshold (e.g., from 4 to 60, or from 10 to 20 breaths per minute for adult, and from 20 to 40 breaths per minute for a child or infant), controller 120 signals venting module 160 to force air or other gas into cuff inflation line 106c, so as to clear the occlusion.

Module 155 can comprise a pressure regulator and/or a flow meter (not shown). The pressure regulator controls the suctioning vacuum applied by module 155, and the flow meter measures the flow generated by the suctioning operation.

A patient connected to a ventilator requires periodic removal of fluid from the trachea. The traditional practice in hospitals is to disconnect the ventilator from the patient, and to insert through the main lumen of the tracheal tube a retractable catheter 105 which is used to remove the fluids from the trachea. Catheter 105 is introduced into the main lumen 202 of device 102, such that its distal end is positioned beyond the distal end 214 of device 102 toward the lungs (according standard of care 1-2 cm above the carina). The proximal end of line 105 is connected to one of the fluid distribution ports of device 10 (e.g., port 16d).

Main controller 120 can synchronize the suction operation with the breathing cycle of the subject and/or the tracheal pressure, wherein module 155 performs the deep suctioning operation through line 105 based on data indicative of the breathing cycle and/or tracheal pressure. Main controller 120 can signal module 155 to perform the deep suctioning operation during the exhale phase of the breathing cycle. Main controller 120 can dynamically update the under-pressure applied by module 155, responsively to the cuff pressure drop during exhale. In an embodiment of the invention, main controller 120 adapts the under-pressure such that the resulting suctioning force is maintained generally constant (e.g., within 20%) throughout the deep suction operation. Main controller 120 can adapt the under-pressure such that the effective under-pressure at the distal end of line 105 is from about 0 mmHg to about 300 mmHg during the entire suctioning phase. This can be done by applying pulsating high vacuum levels, for example, every several breathing cycles.

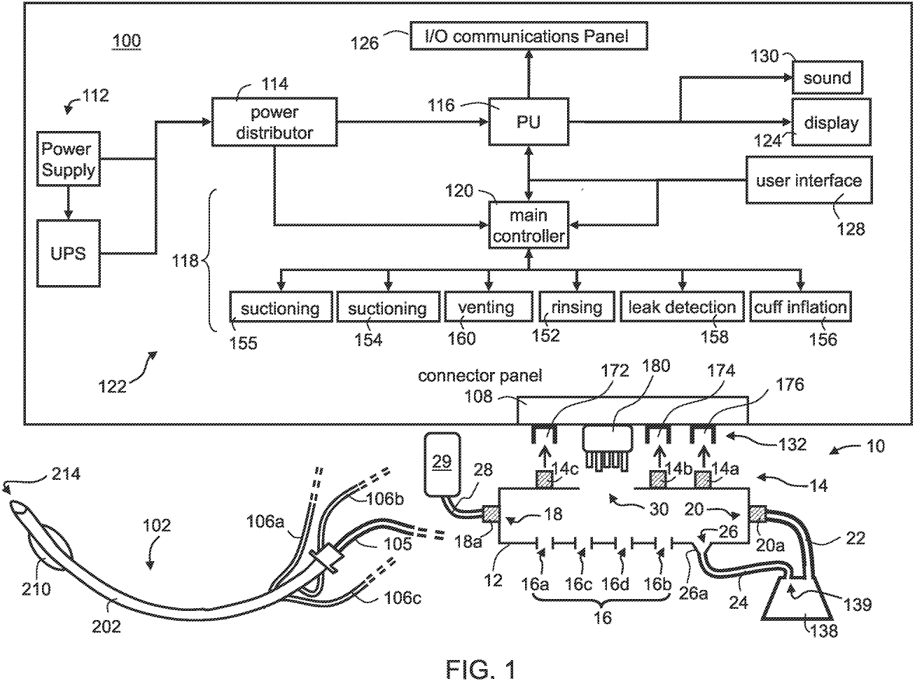

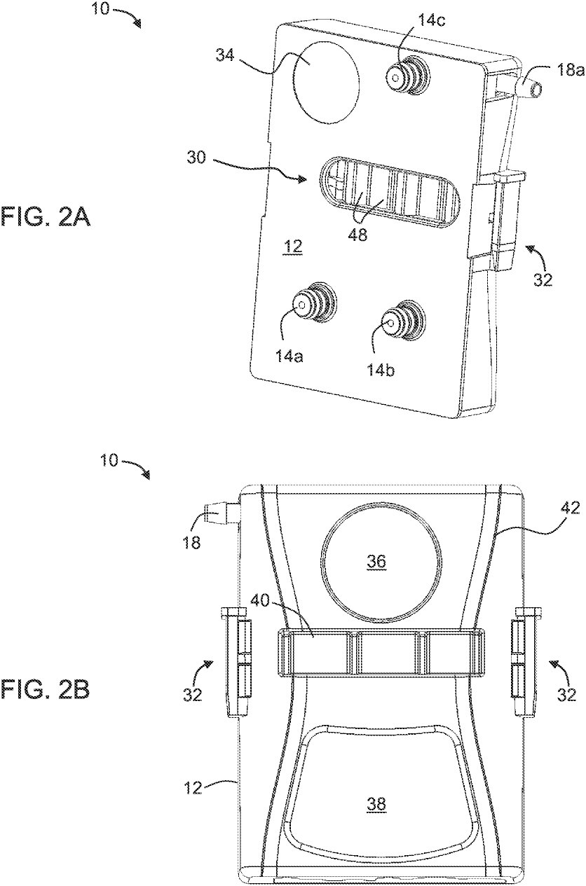

Following is a more detailed description of the principles and operations of fluid distribution device 10, according to some embodiments of the present invention, with reference to FIGS. 2A-7M.

FIGS. 2A and 2B are schematic illustrations of perspective external views of a back side (FIG. 2A) and a front side (FIG. 2B) of fluid distribution device 10, according to some embodiments of the present invention. The back side is connectable to a connector panel of a control system. In embodiments in which fluid distribution device 10 is used during ventilation, such as, but not limited to, the back side can be connectable to the connector panel 108 of system 100, such that the disposable connectors 14 are connected to the panel connectors 132 as further detailed hereinabove.

Fluid distribution device 10 optionally and preferably comprises an enclosure 12, from which the disposable connectors 14 optionally protrude. Optionally and preferably a disposable connector 18a leading to saline inlet port 18 also protrudes out of enclosure 12. Enclosure 12 can be made openable with one or more clamps 32, e.g., snap action clamps for opening and closing disposable enclosure 12. Optionally and preferably, but not necessarily, enclosure 12 is provided with reinforcing ribs 40 to further ensue resistance of the device to pressure. In some embodiments of the present invention enclosure 12 is shaped to allow easy grip, as illustrated by line 42 of FIG. 2B. Preferably, enclosure 12 is disposable. Enclosure 12 is optionally and preferably provided with window 30 through which through which valve system 180 engages and controls fluid interconnectors in device 10, as described in below.

Enclosure 12, can optionally and preferably be formed with dedicated regions 34, 36, 38, for attaching additional components thereto, such as, but not limited to, an instruction sticker, a logo, and the like. Optionally and preferably, device 10 comprises an identification tag (not shown) which can be attached to one or more of dedicated regions 34, 36, 38. The identification tag can be of any machine-readable type known in the art, such as, but not limited to, a barcode (e.g. a QR tag), an RFID and an RTLS. Preferably, the wall of enclosure 12 is made thinner at dedicated regions 34, 36, 38 to mark their location for the attachment of the aforementioned additional components, and to save on material.

FIG. 3 is a schematic illustration of the interior of device 10, for example, as seen when enclosure 12 is opened. Device 10 preferably comprises a first manifold 44 and a second manifold 46, both can be mounted within enclosure 12. Aforementioned fluid distribution ports 16a-d, and optionally and preferably also vacuum port 26, can be formed in second manifold 46. A connector such as, but not limited to, a barb can be mounted on second manifold 46 at each of ports 16a-d. These connectors are shown at 86a-d respectively. Connectors 86a-d are optionally and preferably within enclosure 12 and are accessible for connections to external fluid lines (e.g., lines of the tracheal tube device 102) by opening enclosure 12 is opened. Alternatively, one or more of connectors 86a-d can protrude out of enclosure 12, similarly to connector 18a.

Manifolds 44 and 46 can optionally be connected to each other via a plurality of fluid interconnectors 48 each being controllable by valve system 180 (not shown, see FIG. 1). Specifically, each of manifolds 44 and 46 can optionally comprises a plurality of interconnection ports and the interconnectors 48 individually connect between the interconnection ports of manifold 44 and the interconnection ports of manifold 46. The interconnection ports of manifold 44 are shown at 72, 74 and interconnection ports of manifold 46 are shown at 76, 78.

Preferably, interconnectors 48 connect between the interconnection ports of the manifolds in a one-to-one manner, namely each of the interconnection ports of manifold 44 is connected to one interconnection port of manifold 46, so that the interconnection ports are paired among the manifolds. For example, a barb can be mounted on each of the ports of manifold 44 and 46, wherein the fluid interconnections 48 are shape-wise and size-wise compatible wherein the barbs, and wherein each of fluid interconnection 48 connects between a barb of manifold 44 and a barb of manifold 46. The barbs of manifolds 44 and 46 are generally shown at 54 and 56, respectively. Shown in FIG. 3, are five interconnectors and interconnect ports for each manifold, but other numbers of interconnectors and interconnect ports are also contemplated.

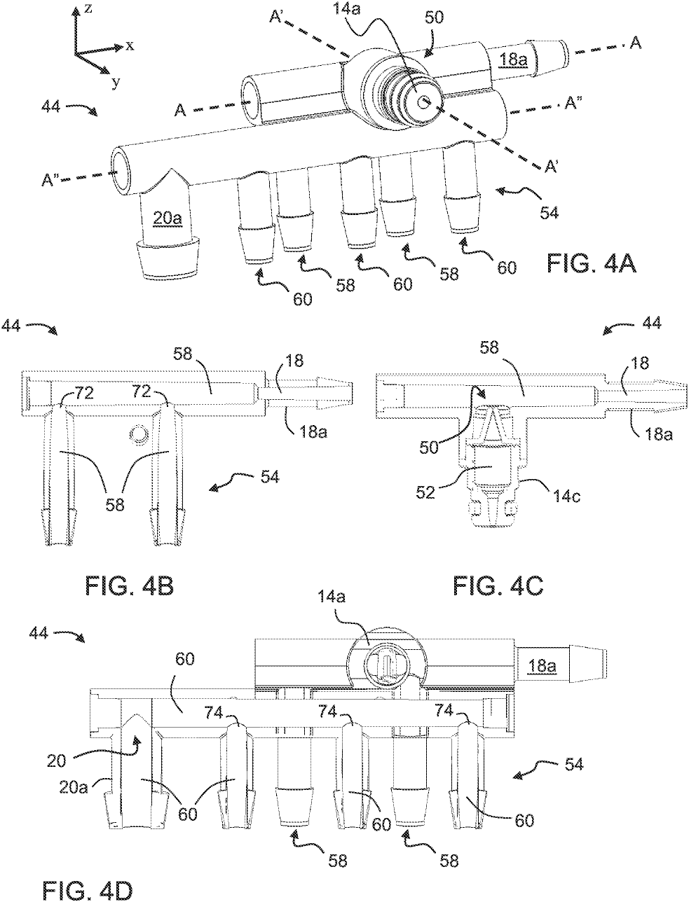

FIGS. 4A-D schematically illustrate first manifold 44 in accordance with some embodiments of the present invention and in greater detail, where FIG. 4A shows an isometric view, and FIGS. 4B-D show cross-sectional views in the x-z plane (FIGS. 4B and 4D) and the x-y plane (FIG. 4C), along the lines A---A (FIG. 4B), A′---A′ (FIG. 4C) and A″---A″ (FIG. 4D) of FIG. 4A. First manifold 44 preferably comprises a fluid inlet 50 for receiving fluid from one of panel connectors. For example, disposable connector 14c can be mounted on fluid inlet 50, in which case inlet 50 receives fluid (e.g., air) from panel connector 172 (see FIG. 1). In embodiments in which device 10 comprises saline inlet port 18 and/or waste port 20, these ports can be also formed on first manifold 44, with a respective connector 18a, 20a, optionally and preferably disposable connectors, mounted thereon.

Manifold 44 preferably comprises two of more separate fluid channels therein. In the schematic illustration shown in FIGS. 4A-D, which is not to be considered as limiting, manifold 44 includes five interconnect ports, of which two interconnect ports, shown at 72, are parts of a first fluid channel 58, and three interconnect ports, shown at 74, are parts of a second fluid channel 60. Preferably, any fluid communication between first 58 and second 60 channels within manifold 44 is prevented. The separation between channels 58 and 60 ensures that there is no mixing in manifold 44 between fluids delivered by different external lines (e.g., to and from the tracheal tube device 102).

First fluid channel 58 is in communication with inlet 50 and 18 (when employed) and therefore interconnect ports 72 serve as efflux ports for delivering fluids (e.g., air, saline) entered to channel 58 from inlet 50 or 18, out of manifold 44. Preferably, but not necessarily, a one-way valve 52 is mounted within disposable fluid connector 14c. The advantage of these embodiments is that it prevents saline from port 18 to enter system 100 through gas supply connector 172.

Interconnect ports 74 of second fluid channel 60 preferably serve for receiving fluids (e.g., air, saline, secretions) into manifold 44. In some embodiments of the present invention, waste port 20 is within, or otherwise in fluid communication with, second fluid channel 60. Since waste port 20 is connected to waste collection container 138, the under-pressure in container 138 also exists in waste port 20, and so the influx through interconnect ports 74 into channel 60 can be established by virtue of the under-pressure in waste port 20.

Some of the interconnection ports of second manifold 46 serve as influx interconnection ports 78 through which fluid enters manifold 46 from channel 58 of manifold 44, and the other interconnection ports serve as efflux interconnection ports 76 through which fluid exits manifold 46 into channel 60 of manifold 44. In embodiments in which the interconnectors 48 connect between the interconnection ports of the manifolds in a one-to-one manner, the number of interconnection ports in manifolds 44 and 46 is the same, wherein each efflux interconnection port of manifold 44 is connected via an interconnector to an influx interconnection port of manifold 46, and each influx interconnection port of manifold 44 is connected via an interconnector to an efflux interconnection port of manifold 46.

In some embodiments of the present invention second manifold 46 is structured to established separate flow paths to two or more, more preferably three or more different distribution ports. Preferably, but not necessarily, second manifold 46 establishes a separate flow path for each of the distribution ports.

Flow paths that are established within manifold 46 preferably do not form a one-to-one mapping between the distribution ports and the interconnection ports. That is to say, there is at least one distribution port that is in fluid communication with more than one interconnection port. For example, a particular fluid distribution port (e.g., at least one of ports 16a and port 16b) can be in fluid communication with one influx interconnection port 78 and one efflux interconnection port 76 of second manifold 46. The advantage of this embodiment is that it allows the same fluid distribution port to deliver as well to extract fluids from the tracheal tube device 102. In various exemplary embodiments of the invention there is also at least one distribution port that is fluidly separated within manifold 46 from all the interconnection ports. For example, when port 16c is in direct fluid communication with disposable fluid connector 14b that connects to cuff inflation connector 174, port 16c can be fluidly separated within manifold 46 from all interconnection ports 16a-d as well as from vacuum port 26.

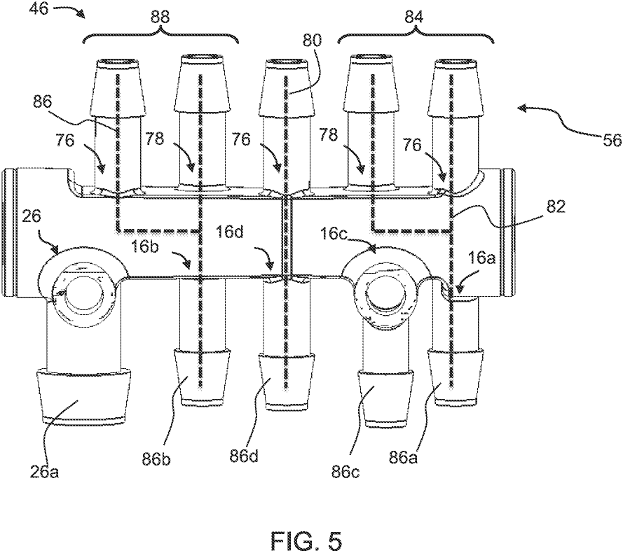

A representative example of separate flow paths within manifold 46, according to some embodiments of the present invention, is schematically illustrated in FIG. 5. The separate fluid paths are illustrated in FIG. 5 by dashed lines. As shown, a first fluid path 82 is a bifurcated path between a first pair 84 of fluid interconnectors 76, 78 and a first distribution port 16a, a second fluid path 86 is a bifurcated path between a second pair 88 of fluid interconnectors of fluid interconnectors 76, 78 and a second distribution port 16b, and a third fluid path 80 is a non-furcated path that establishes a one to-one fluid communication between one fluid interconnector 76 and one distribution port 16d. Preferably, first and second fluid paths 84 and 88 are dedicated to deliver or extract fluids to or from fluid lines 106a, 106b, but not to or from cuff inflation line 106c, of tracheal tube device 102, and third path 80 is dedicated to extract fluids from retractable catheter 105.

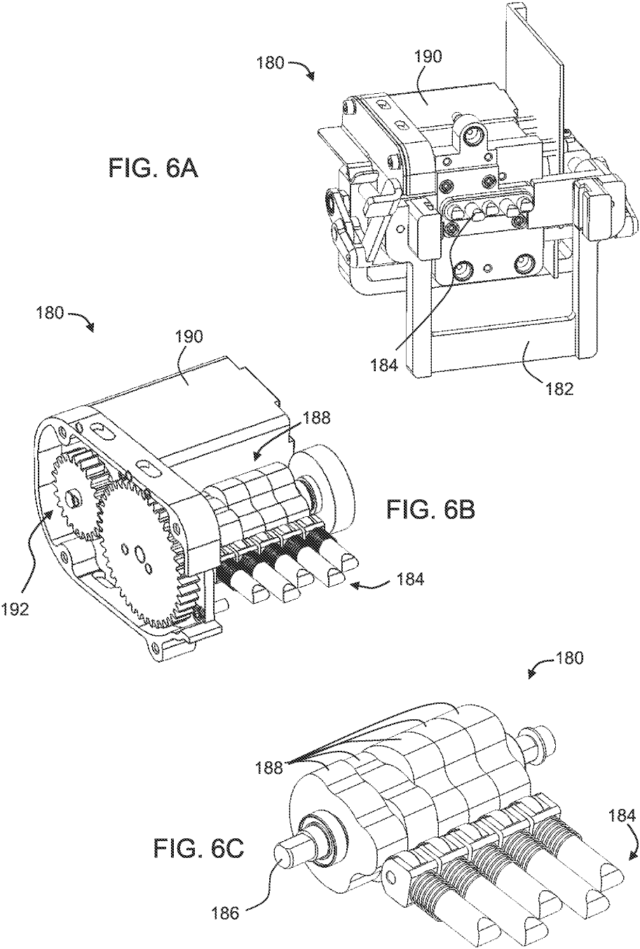

Reference is now made to FIGS. 6A-C which are schematic illustrations of valve system 180, according to various exemplary embodiments of the present invention. Valve system 180 is particularly useful for controlling the flow in fluid interconnectors 48 of device 10, but can alternatively be used with any system that requires controlling a flow through a tubing system having fluid interconnectors, without requiring an opening or junction in the tubing system, and without forming direct contact with the fluid contained in the tubing. Valve system 180 is capable of controlling flow in tubes having flexible walls. Thus, when valve system 180 is employed for controlling flow in a fluid interconnector, the fluid interconnector is a flexible-wall tube. Specifically, when valve system 180 is employed for controlling the flow in fluid interconnectors 48 of device 10, the wall of each of the fluid interconnectors 48 is made flexible and compressible.

Valve system 180 typically comprises a body structure 182 on which various components of valve system 180 can be mounted. When valve system 180 is used with system 100 for controlling the flow in fluid interconnectors 48 of device 10, body structure 182 of system 180 can be mounted on system 100 opposite to window 30 of enclosure 12 (see FIG. 1). Valve system 180 preferably comprises a plurality of movable pressing members 184, which are typically, but not necessarily, shaped as pressing fingers. Each of pressing members 184 is aligned to engage a wall of one of interconnectors 48 (not shown, see FIG. 3), such that a motion of a pressing member toward a respective wall of the interconnector generates a compressive force on the respective wall and restricts or ceases the flow through the respective interconnector.

The motion of pressing members 184 is preferably a reciprocal linear motion. This can be achieved by providing valve system 180 with a cam shaft 186 having a plurality of cams 188, and being rotatable, for example, by means of a motor 190 and optionally and preferably a transmission gear 192. Motor 190 is preferably controllable by controller 120 of system 100, in response to signals received from processing system 116 to execute the various procedures. Each of cams 188 is aligned with one of pressing members 184 in a manner that rotation of the cam 188 establishes a reciprocal linear motion of the pressing member 184. Alternatively, the motion of pressing members 184 can be controlled by applying other type of forces. For example, one or more of members 184 can be a pneumatically, electrically or electromagnetically driven piston. While the embodiments below are described with a particular emphasis to pressing members 184 that are controlled by a cam shaft, it is to be understood that other types of mechanisms for controlling pressing members 184, such as, but not limited to, the aforementioned pneumatic, electric, or electromagnetic mechanisms, is also contemplated according to some embodiments of the present invention.

In use with system 100 and device 10, controller 120 signals motor 190 to rotate shaft 186 such that pressing members 184 selectively and individually restrict (e.g., block) or allow the flow in interconnectors 48, thereby closing and opening fluid paths between the distribution ports 16 and the connectors on panel 108, and optionally and preferably also between distribution ports 16 and the saline inlet port 18, and/or the waste port 20, in accordance with the procedures performed by controller 120.

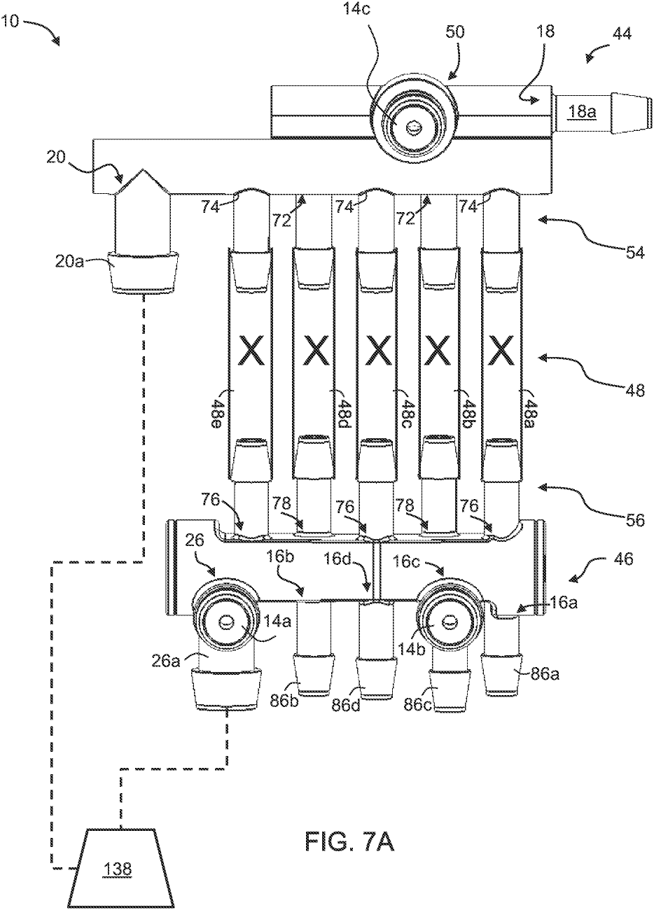

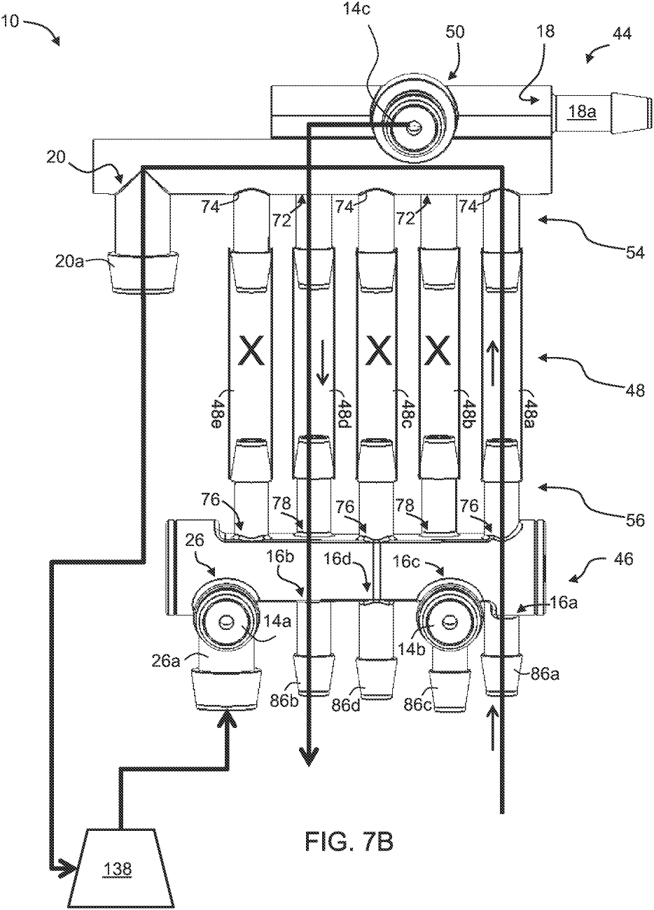

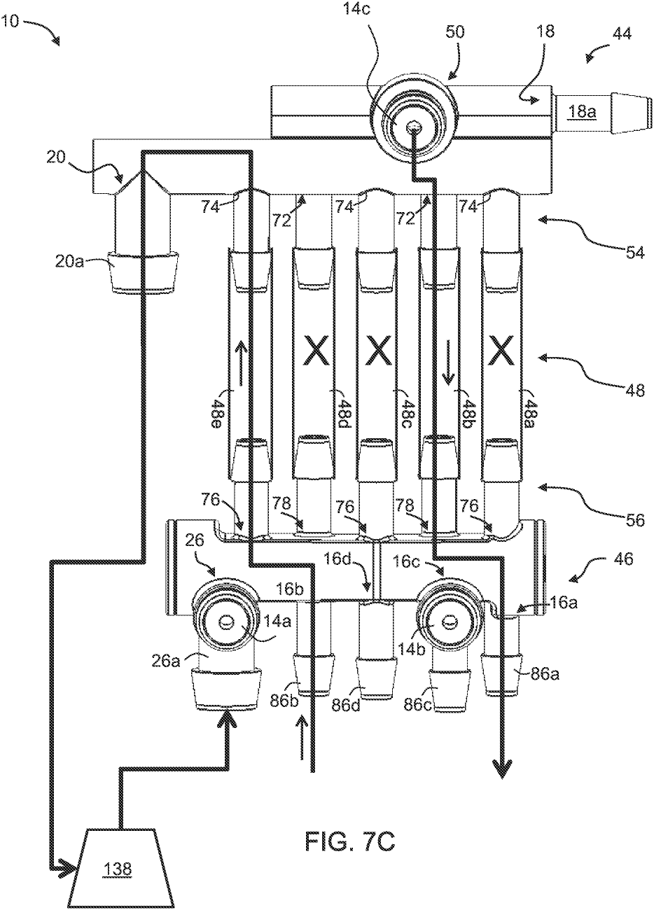

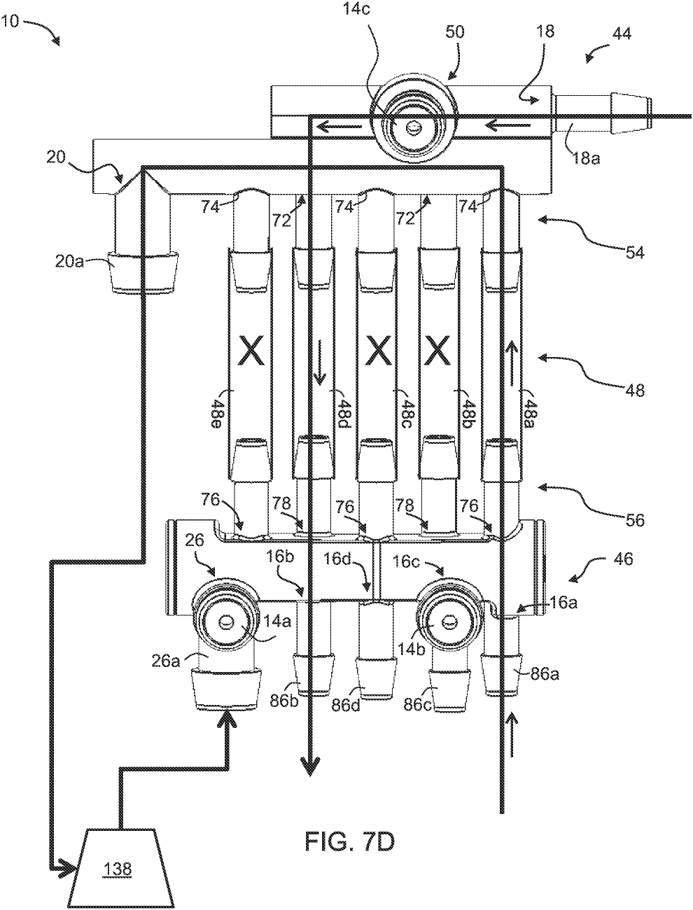

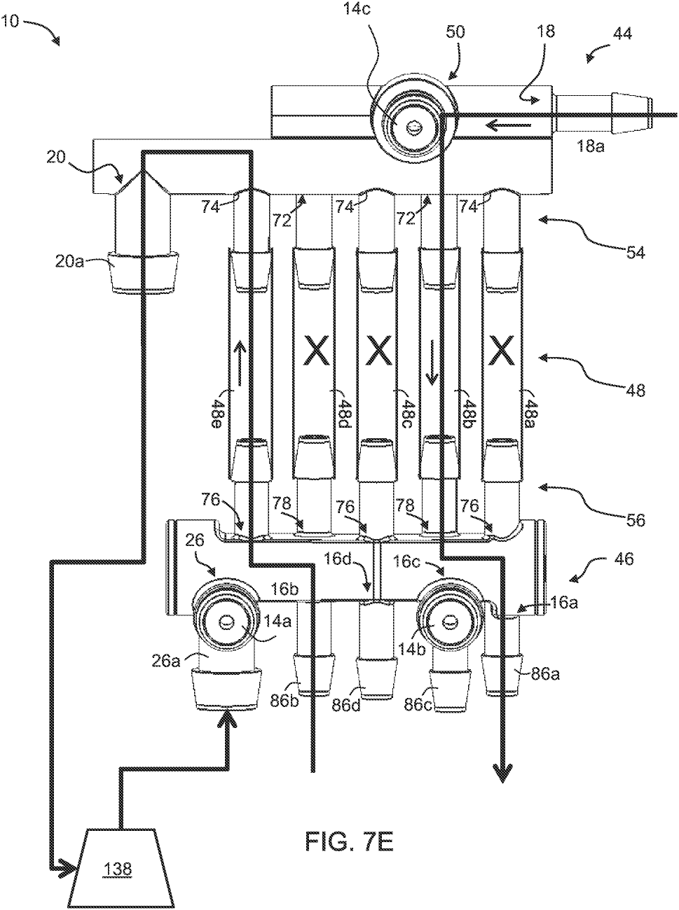

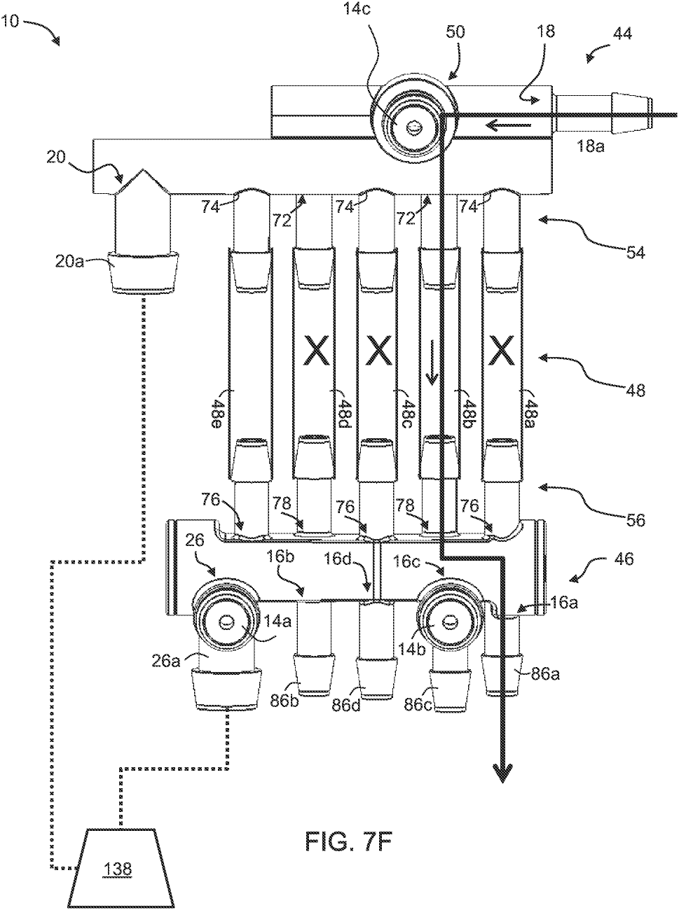

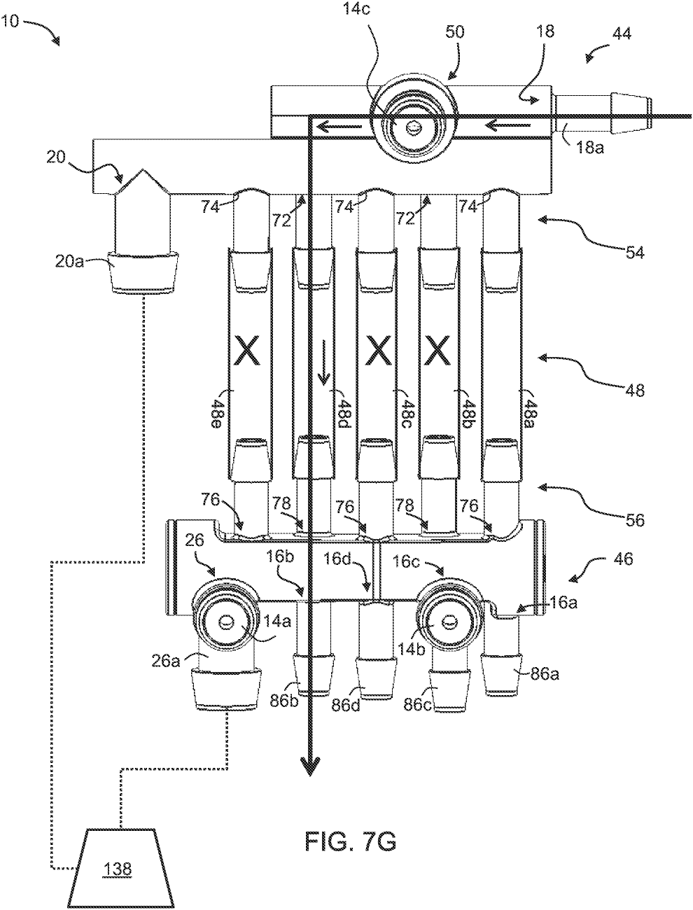

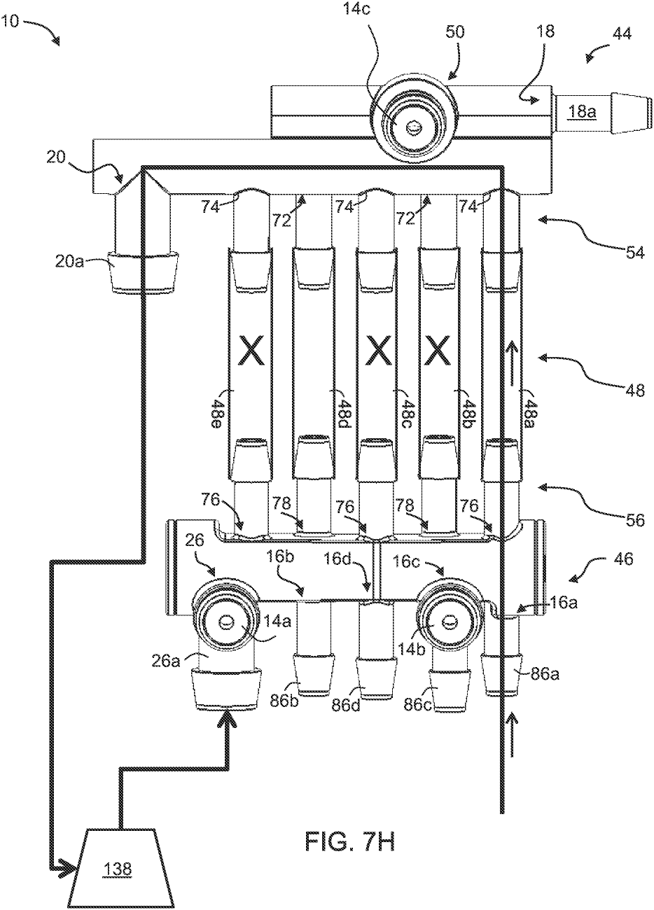

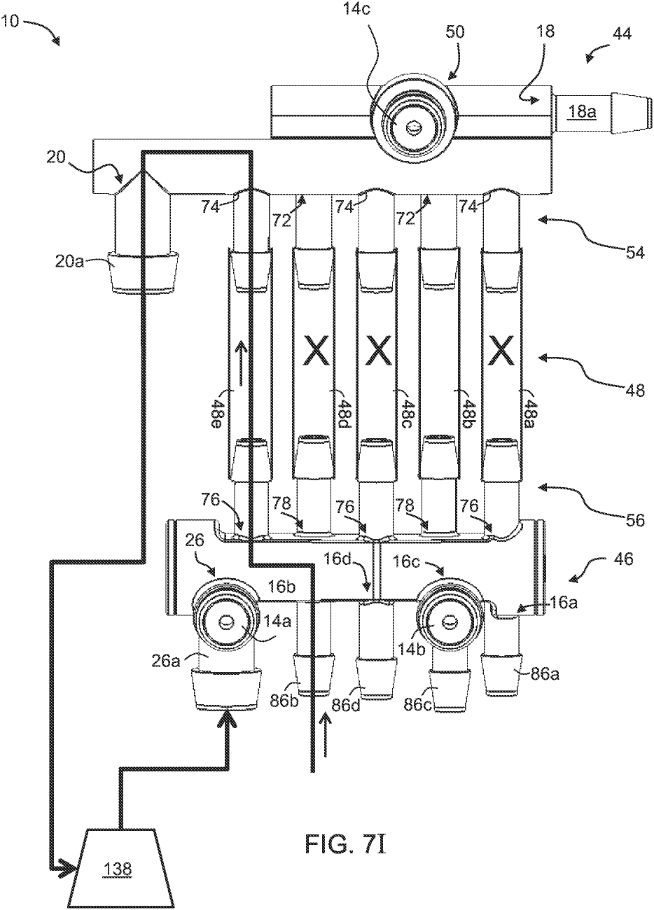

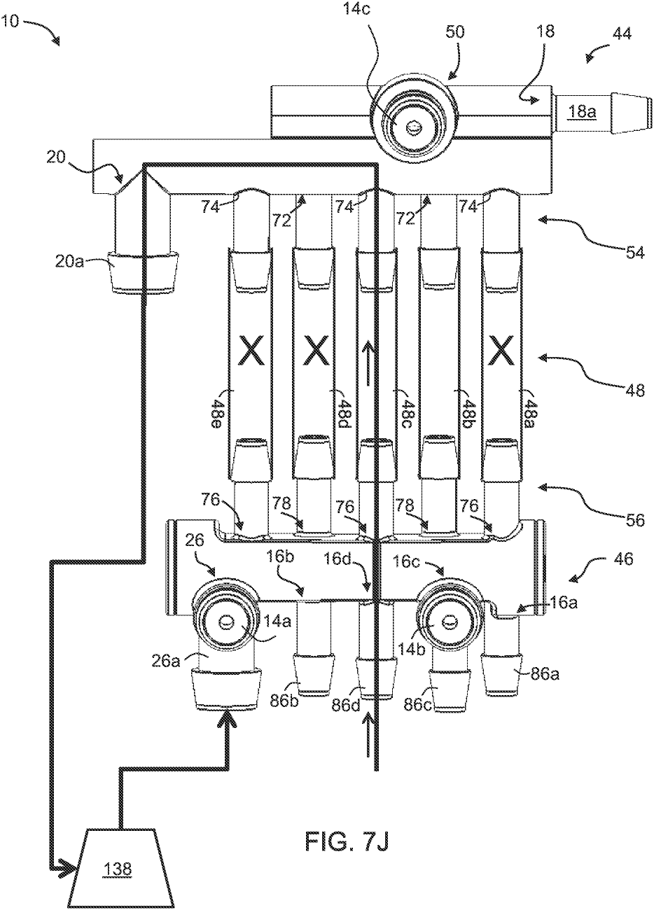

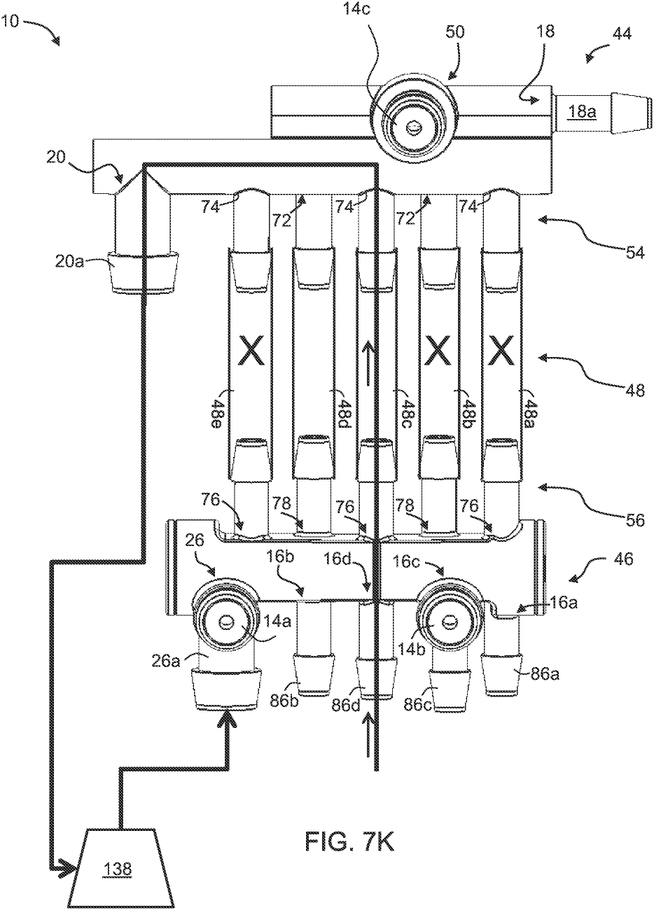

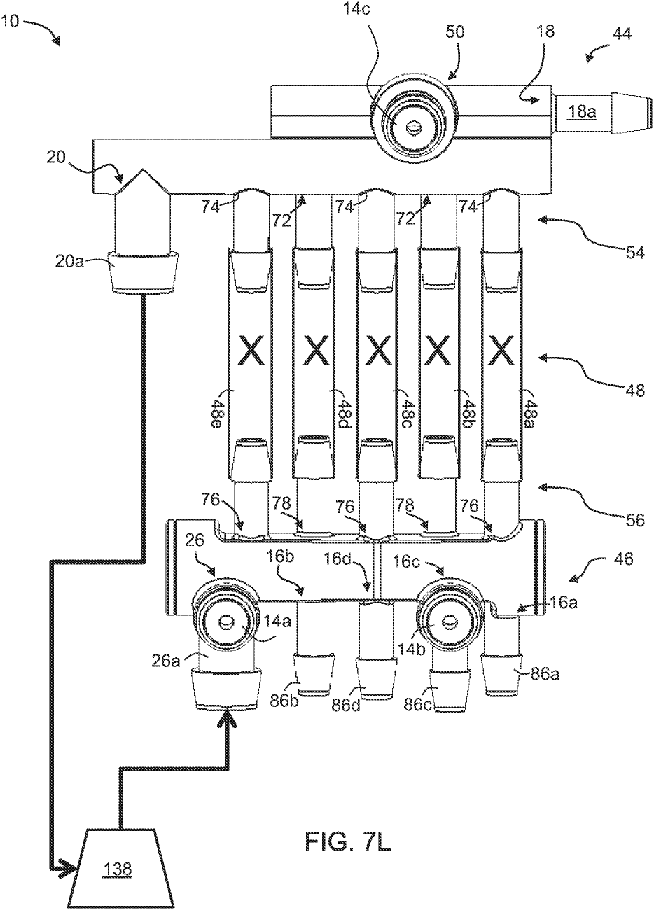

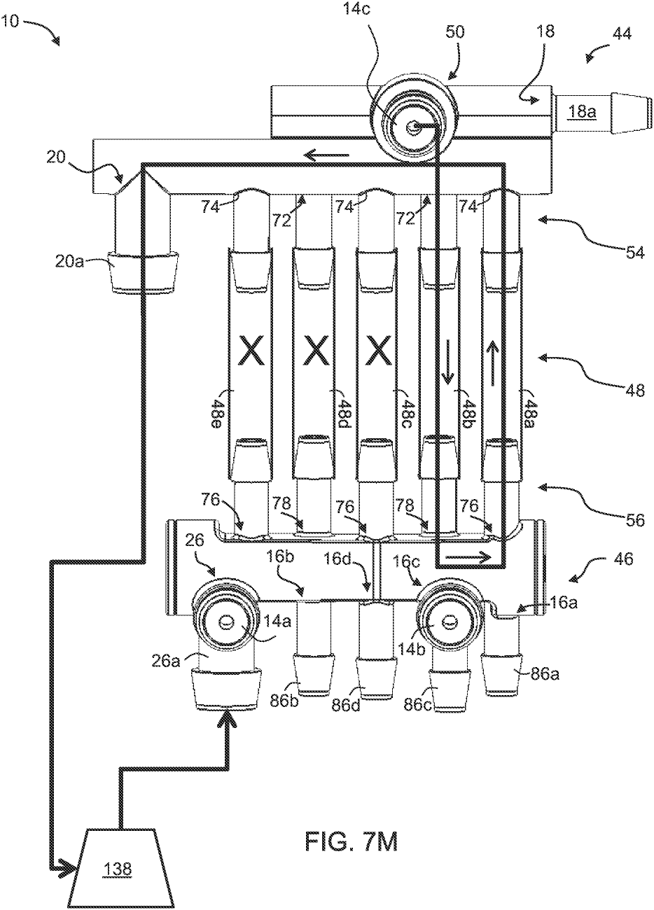

Several examples of such fluid paths, in accordance with some embodiments of the present invention, will now be described with reference to FIGS. 7A-M. In FIGS. 7A-M, an “X” symbol in a fluid interconnector 48 represents a state in which valve system 180 blocks a flow in the fluid interconnector (for example, by pressing the respective member 184 against the wall of the interconnector); a dashed line between connector 20a and waste collection container 138, or between connector 20a and vacuum port connector 26a represents a state in which there is no flow to and from container 138; solid lines represent open fluid path, and arrow represent flow direction along the respective fluid path. The examples relate to a configuration with five fluid interconnector 48, designated by reference signs 48a, 48b, 48c, 48d and 48e.

FIG. 7A illustrates a standby state in which all the fluid paths are closed and there is also no flow to and from container 138.

FIG. 7B illustrates a state in which interconnectors 48a and 48d are opened, and all other interconnectors are closed. Fluid enters manifold 46 through port 16a, continues through interconnectors 48a to enter manifold 44 through an influx interconnect port 74, and is redirected through waste port 20 into container 138 by means of the under pressure delivered by vacuum port 26. Gas (e.g., air) delivered by system 100 via gas supply connector 172 enters manifold 44 through port 50, exits manifold 44 through one of the efflux ports 72, continues in interconnector 48d, enters manifold 46 through one of the influx ports 78, and exit manifold 46 through port 16b. This state is particularly useful for evacuation of tracheal secretions via line 106a while forcing air via line 106b of tracheal tube device 102.

FIG. 7C illustrates a state in which interconnectors 48b and 48e are opened, and all other interconnectors are closed. Fluid enters manifold 46 through port 16b, continues through interconnectors 48e to enter manifold 44 through an influx interconnect port 74, and is redirected through waste port 20 into container 138 by means of the under pressure delivered by vacuum port 26. Gas (e.g., air) delivered by system 100 via gas supply connector 172 enters manifold 44 through port 50, exits manifold 44 through one of the efflux ports 72, continues in interconnector 48b, enters manifold 46 through one of the influx ports 78, and exits manifold 46 through port 16a. This state is particularly useful for evacuation of tracheal secretions via line 106b while forcing air via line 106a of tracheal tube device 102.

FIG. 7D illustrates a state in which interconnectors 48a and 48d are opened, and all other interconnectors are closed. Fluid enters manifold 46 through port 16a, continues in interconnectors 48a to enter manifold 44 through one of the influx interconnect ports 74, and is redirected through waste port 20 into container 138 by means of the under pressure delivered by vacuum port 26. Saline delivered by saline source 29 enters manifold 44 through port 18, exits manifold 44 through one of the efflux ports 72, continues in interconnector 48d, enters manifold 46 through one of the influx ports 78, and exits manifold 46 through port 16b. This state is particularly useful for evacuation of tracheal secretions via line 106a while rinsing by saline via line 106b of tracheal tube device 102.

FIG. 7E illustrates a state in which interconnectors 48b and 48e are opened, and all other interconnectors are closed. Fluid enters manifold 46 through port 16b, continues in interconnectors 48e to enter manifold 44 through one of the influx interconnect ports 74, and is redirected through waste port 20 into container 138 by means of the under pressure delivered by vacuum port 26. Saline delivered by saline source 29 enters manifold 44 through port 18, exits manifold 44 through one of the efflux ports 72, continues in interconnector 48b, enters manifold 46 through one of the influx ports 78, and exits manifold 46 through port 16a. This state is particularly useful for evacuation of tracheal secretions via line 106b while rinsing by saline via line 106a of tracheal tube device 102.

FIG. 7F illustrates a state in which interconnector 48b is opened, interconnectors 48a, 48c and 48d are closed, and no vacuum is delivered by port 26 so that there is no flow to and from container 138. Interconnector 48e can also be closed but since there is no flow to container 138, it can also remain opened, as illustrated in FIG. 7F. Saline delivered by saline source 29 enters manifold 44 through port 18, exits manifold 44 through one of the efflux ports 72, continues in interconnector 48b, enters manifold 46 through one of the influx ports 78, and exits manifold 46 through port 16a. This state is particularly useful for rinsing by saline via line 106a of tracheal tube device 102.

FIG. 7G illustrates a state in which interconnector 48d is opened, interconnectors 48b, 48c and 48e are closed, and no vacuum is delivered by port 26 so that there is no flow to and from container 138. Interconnector 48a can also be closed but since there is no flow to container 138, it can also remain opened, as illustrated in FIG. 7G. Saline delivered by saline source 29 enters manifold 44 through port 18, exits manifold 44 through one of the efflux ports 72, continues in interconnector 48d, enters manifold 46 through one of the influx ports 78, and exits manifold 46 through port 16b. This state is particularly useful for rinsing by saline via line 106b of tracheal tube device 102.

FIG. 7H illustrates a state in which interconnector 48a is opened, interconnectors 48b, 48c and 48e are closed, and no gas or saline are delivered into manifold 44. Interconnector 48d can also be closed but since gas or saline are delivered, it can also remain opened, as illustrated in FIG. 7H. Fluid enters manifold 46 through port 16a, continues in interconnectors 48a to enter manifold 44 through one of the influx interconnect ports 74, and is redirected through waste port 20 into container 138 by means of the under pressure delivered by vacuum port 26. This state is particularly useful for executing a leak detection procedure via line 106a of tracheal tube device 102.

FIG. 7I illustrates a state in which interconnector 48e is opened, interconnectors 48a, 48c and 48d are closed, and no gas or saline are delivered into manifold 44. Interconnector 48b can also be closed but since gas or saline are delivered, it can also remain opened, as illustrated in FIG. 7I. Fluid enters manifold 46 through port 16b, continues in interconnectors 48e to enter manifold 44 through one of the influx interconnect ports 74, and is redirected through waste port 20 into container 138 by means of the under pressure delivered by vacuum port 26. This state is particularly useful for executing a leak detection procedure via line 106b of tracheal tube device 102.

FIGS. 7J and 7K illustrates a state in which interconnector 48c is opened, interconnectors 48a, 48d and 48e (FIG. 7J) or 48a, 48b and 48e (FIG. 7K) are closed, and no gas or saline are delivered into manifold 44. In FIG. 7J, interconnector 48b can also be closed but since gas or saline are delivered, it can also remain opened. In FIG. 7K, interconnector 48d can also be closed but since gas or saline are delivered, it can also remain opened. Fluid enters manifold 46 through port 16d, continues in interconnectors 48e to enter manifold 44 through one of the influx interconnect ports 74, and is redirected through waste port 20 into container 138 by means of the under pressure delivered by vacuum port 26. This state is useful for more than one operation.

In some embodiments of the present invention this state is used for executing a leak detection procedure via line 106a of tracheal tube device 102.

In some embodiments of the present invention this state is used for executing evacuation of secretions using separate suction catheter 105, the proximal end of which being connected to connector 86d and the distal end of which being introduced to the main lumen of device 102.

In some embodiments of the present invention this state is used for removing contaminated gas out of container 138, for example, gas originates from the trachea of the subject and that is accumulated in container 138 while system 100 applies suction in one or more of the lines 106a of tracheal tube device 102. Removal of contaminated gas out of container 138, can be done by allowing clean gas to enter manifold 46 through port 16d. Thus, rather than establishing fluid communication between connector 86d and the main lumen of device 102, this embodiment establishes fluid communication between connector 86d and ambient air, or a source of clean gas (not shown). By means of the under pressure delivered by vacuum port 26, the clean gas or ambient air flows through connector 86d, enters manifold 46, continues to manifold 44, redirects into container 138, and replaces at least a portion of the contaminated gas therein.

FIG. 7L illustrates a state in which all interconnectors are closed, and no gas or saline are delivered into manifold 44. This state is particularly useful for checking whether there is a leak in container 138.

FIG. 7M illustrates a state in which interconnectors 48a and 48b are opened, and all other interconnectors are closed. Gas (e.g., air) delivered by system 100 via gas supply connector 172 enters manifold 44 through port 50, exits manifold 44 through one of the efflux ports 72, continues in interconnector 48b, enters manifold 46 through one of the influx ports 78, redirected back to manifold 44 through one of the efflux ports 76 and interconnector 48a to enter manifold 44 through one of the influx interconnect ports 74, and is redirected through waste port 20 into container 138 by means of the under pressure delivered by vacuum port 26. This state is particularly useful for forcing air to container 138 for the purpose of venting the container.

As used herein the term “about” refers to ± 10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first, indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A fluid distribution device for mechanical ventilation, comprising:
a disposable enclosure;
disposable fluid connectors connectable to compatible panel connectors of a control system for controlling flow in a tracheal tube device; and
a first manifold and a second manifold, both mounted within said disposable enclosure and being connected to each other via a plurality of fluid interconnectors each being controllable by a valve system;
wherein said first manifold comprises a fluid inlet for receiving fluid from one of said panel connectors, and wherein said second manifold comprises a plurality of fluid distribution ports adapted for establishing fluid communications between said fluid interconnectors and fluid lines of said tracheal tube device.

2. The device of claim 1, wherein said second manifold is structured to established separate flow paths to at least two different distribution ports.

3. The device of claim 2, wherein said separate flow paths comprise a first fluid path between a first pair of fluid interconnectors and a first distribution port, and a second fluid path between a second pair of fluid interconnectors and a second distribution port.

4. The device of claim 1, wherein said second manifold is structured to established separate flow paths to at least three different distribution ports.

5. (canceled)

6. The device according to claim 1, wherein said disposable fluid connectors comprise a disposable cuff inflation connector connectable to a compatible control panel cuff inflation connector, and wherein said second manifold comprises a cuff inflation port for establishing a fluid communication between said disposable cuff inflation connector and a cuff inflation line of said tracheal tube device.

7. (canceled)

8. The device according to claim 1, wherein said disposable fluid connectors comprise a disposable vacuum connector connectable to a compatible control panel vacuum connector, and wherein said second manifold comprises a vacuum port for establishing a fluid communication between said disposable vacuum connector and a vacuum line generating under pressure in a waste collection container receiving waste fluid from a subject.

9. (canceled)

10. The device according to claim 8, wherein said first manifold comprises a waste port connectable to a waste line delivering said waste fluid to said waste collection container.

11. (canceled)

12. The device according to claim 10, wherein said first manifold comprises therein a first fluid channel and a second fluid channel separated from each other, and wherein said waste port of said first manifold is constituted to be fed by fluid from said second fluid channel, and is fluidly separated within said first manifold from said first fluid channel.

13. (canceled)

14. The device according to claim 1, wherein said first manifold comprises at least two separate fluid channels therein.

15. (canceled)

16. The device according to claim 14, wherein said disposable fluid connectors comprise a disposable fluid inlet connector connectable to a compatible gas supply panel connector of said control system, wherein a first fluid channel is in fluid communication with said disposable fluid inlet connector, and wherein a second fluid channel is fluidly separated within said first manifold from said disposable fluid inlet connector.

17-19. (canceled)

20. The device according to claim 14, comprising a saline inlet port connectable to saline supply line, wherein a first fluid channel is constituted to be fed by saline from said saline inlet port, and wherein a second fluid channel is fluidly separated within said first manifold from said saline inlet port.

21-25. (canceled)

26. The device according to claim 1, wherein said disposable enclosure comprises a window exposing said fluid interconnectors to allow said valve system to engage said fluid interconnectors.

27. (canceled)

28. The device according to claim 1, wherein said valve system comprises a plurality of movable pressing members each aligned to engage a wall of one of said fluid interconnectors, such that a motion of a pressing member toward a respective wall generates a compressive force on said respective wall and restricts or ceases flow through a respective fluid interconnector.

29. (canceled)

30. The device of claim 28, wherein said valve system comprises a cam shaft having a plurality of cams each aligned with one of said pressing members in a manner that rotation of a cam establishes a linear motion of a respective pressing member.

31. (canceled)

32. A method of distributing fluid within a tracheal tube device, the method comprising:
connecting the device according to claim 1 to a control system; and
operating said controller of said control system to transmit control signals to said valve system.

33. (canceled)

34. A system for controlling and monitoring flow in a cuffed tracheal tube device, comprising:
a connector panel, adapted for connecting to a disposable enclosure of a fluid distribution device, and having a plurality of panel connectors adapted for connecting to disposable fluid connectors of said fluid distribution device;
a valve system, configured for selectively controlling flow within said fluid distribution device; and
a controller configured to transmit control signals to said valve system;
wherein said fluid distribution device comprises a first manifold and a second manifold, both mounted within said disposable enclosure and being connected to each other via a plurality of fluid interconnectors each controllable by said valve system, wherein said first manifold comprises a fluid inlet for receiving fluid from one of said panel connectors, and wherein said second manifold comprises a plurality of fluid distribution ports adapted for establishing fluid communications between fluid interconnectors and fluid lines of said tracheal tube device.

35. A method of distributing fluid within a tracheal tube device, the method comprising:
connecting a fluid distribution device to a control system controlling flow in a tracheal tube device; and
operating a controller of said control system to transmit control signals to a valve system controlling flow within said fluid distribution device;
wherein said fluid distribution device comprises:
a disposable enclosure;
disposable fluid connectors connectable to compatible panel connectors of said control system; and
a first manifold and a second manifold, both mounted within said disposable enclosure and being connected to each other via a plurality of fluid interconnectors each being controllable by said valve system, wherein said first manifold comprises a fluid inlet for receiving fluid from one of said panel connectors, and wherein said second manifold comprises a plurality of fluid distribution ports adapted for establishing fluid communications between fluid interconnectors and fluid lines of said tracheal tube device.

36-41. (canceled)

42. The method according to claim 35, wherein said control signals comprise a signal causing said valve system to open a fluid path from a fluid distribution port to a waste port.

43. (canceled)

44. The method of claim 42, wherein said signal also causes said valve system to simultaneously open another fluid path from a panel connector of said control system to another fluid distribution port.

45. (canceled)

46. The method according to claim 35, wherein said control signals comprise a signal causing said valve system to open a fluid path from a panel connector of said control system to one of said interconnectors and from said interconnector to a waste port through another interconnector.

47-63. (canceled)
